(19) United States
(12) Patent Application Publication
(10) Pub. No.: US 2008/0140054 A1
(43) Pub. Date: Jun. 12, 2008

(54) Rotating Means For Suction Tube

(76) Inventor: George Lee, Taipei (TW)

Correspondence Address: HDSL, 4331 STEVENS BATTLE LANE, FAIRFAX, VA, 22033, US

(21) Appl. No.: 11/567,838

(22) Filed: Dec. 7, 2006

(51) Int. Cl. A61M 25/18

(52) U.S. Cl. 604/533

(57) ABSTRACT

A rotating means for a suction tube is capable of adjusting the position of the suction valve. Such means is mainly made of a suction valve. The interior of the suction valve is in communication with each other. The rear end of the suction valve has a first connecting tube. Further, a second connecting tube is provided at the front end of the suction valve opposing the first connecting tube. The interior of the second connecting tube is connected to a rotating tube. The outer periphery of the rotating tube is provided with a resilient protruding ring, so that a gap is formed between the rotating tube and the inner periphery of the second connecting tube. Finally, the second connecting tube is connected to a sleeve, and the sleeve is then connected to the outer periphery of the rotating tube. With the gap formed between the rotating tube and the second connecting tube, the rotating tube and the second connecting tube can be displaced with respect to each other, thereby to adjust and rotate the position of the suction tube.

### BACKGROUND OF THE INVENTION

1. Field of the Invention

The present invention relates to a suction tube, and in particular to a rotatable suction tube.

2. Description of Prior Art

During a general medical procedure, in view of safety, doctor always use a suction tube to remove the sputa existed in the respiratory tract of the patients who cannot remove the sputa by themselves, thereby to keep the respiratory tract unobstructed.

The current suction tube is used to facilitate the removing of the sputa of the patient, and in addition, it can introduce oxygen into the respiratory tract of the patient by being connected to an oxygen-delivering tube, thereby to help the patient to keep breathing. Therefore, in order to combine the suction of sputa and the function of facilitating breathing, the conventional suction tube is provided with a suction valve having a plurality of connecting tubes for connecting the suction tube and the oxygen-delivering tube, or alternatively connecting to the center of other conduits.

In terms of the structure of the existing suction tube, a connecting port of the suction tube is connected and communicated with other conduits, such as the conduit in the respiratory tract of a patient, thereby to perform the suction of sputa or the delivery of oxygen to help the patients who cannot take care of themselves to smooth their respiratory tract. However, in order to adjust the position of the suction tube to correspond to the position of the oxygen-delivering tube, or re-adjust the suction tube to relieve the uncomfortable feeling of the patient, the only way of re-adjusting the position of the suction valve is to first remove all the connecting conduits from the suction valve and then connect different conduits thereon again. Such way of adjusting not only wastes time and labor, but also generates some unnecessary actions that might make the patient in danger. Therefore, it is an important issue for those skilled in this art to overcome the above-mentioned problems.

### SUMMARY OF THE INVENTION

In view of the above drawbacks, the present invention is to provide a rotating means for a suction tube capable of adjusting the position of the suction tube. By providing a rotating tube within the connecting tube to communicate with external conduits, and making a protruding ring provided on the outer circumferential surface of the rotating tube to abut against the inner circumferential surface of the mouth of the suction tube, the rotating tube and the inner circumferential surface of the connecting tube can be displaced with each other, so that the user can easily rotate the suction tube.

In order to achieve the objects, the present invention provides a rotating means for a suction tube. Such means is mainly made of a suction valve. The interior of the suction tube is in communication with each other. The rear end of the suction valve has a first connecting tube. Further, a second connecting tube is provided at front end of the suction tube opposing the first connecting tube. The interior of the second connecting tube is connected to a rotating tube. The outer periphery of the rotating tube is provided with a protruding ring, so that a gap is formed between the rotating tube and the inner edge of the second connecting tube. Finally, the second connecting tube is connected to a sleeve, and the sleeve is then connected to the outer periphery of the rotating tube. With the gap formed between the rotating tube and the second connecting tube, the rotating tube and the second connecting tube can be displaced with respect to each other, thereby to adjust and rotate the position of the suction tube.

### BRIEF DESCRIPTION OF THE DRAWINGS

FIG. 1 is an exploded perspective view of the present invention;

FIG. 2 is a cross-sectional view of the present invention;

FIG. 3 is a schematic view showing the state in which the present invention is completely assembled;

FIG. 4 is an assembled perspective view of the present invention;

FIG. 5 is an assembled cross-sectional view of the present invention; and

FIG. 6 is a schematic view showing the operation of the present invention.

### DETAILED DESCRIPTION OF THE INVENTION

The technical contents and the detailed description of the present invention will be made with reference to the accompanying drawings.

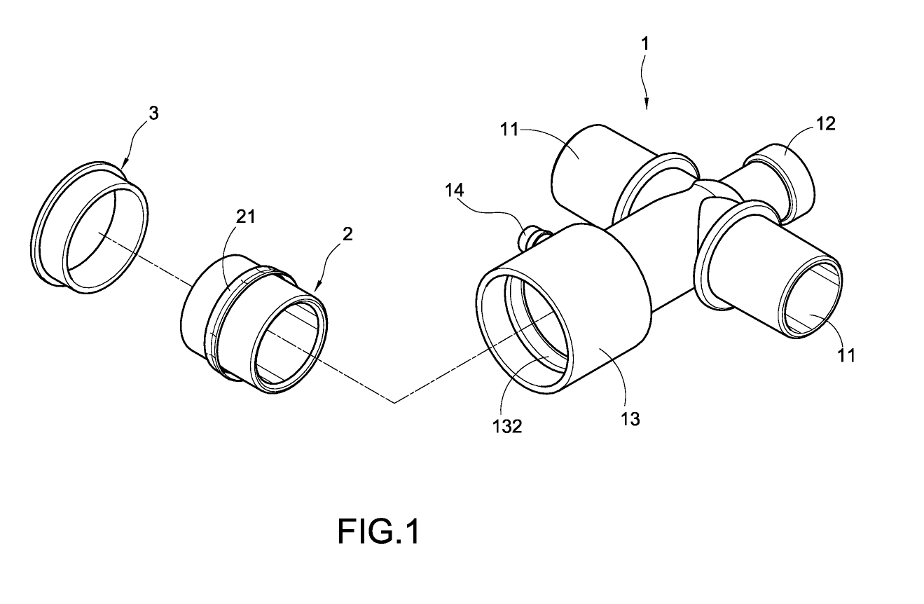

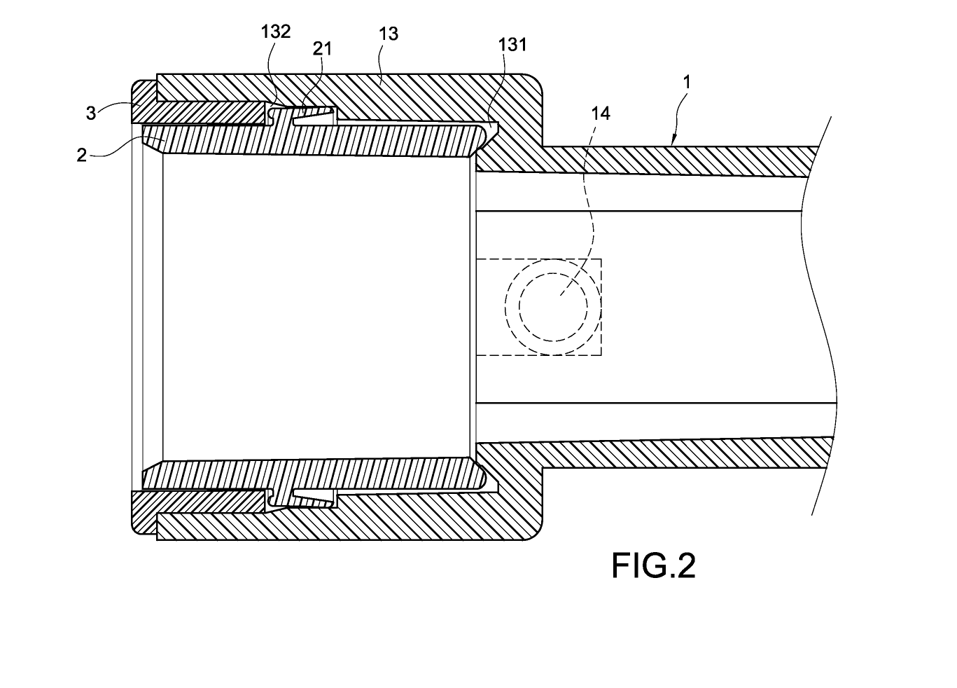

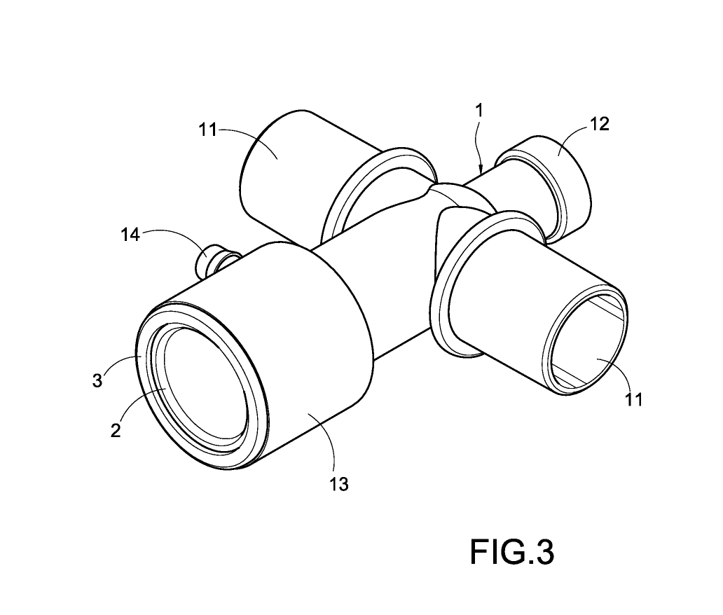

FIG. 1 is an exploded perspective view of the present invention. FIG. 2 is a cross-sectional view of the present invention. FIG. 3 is a schematic view showing the state in which the present invention is completely assembled. As shown in the figures, the suction tube of the present invention is mainly made of a suction valve 1. The profile of the suction valve 1 is formed into a cross. The interior of the suction tube 1 is in communication with each other. Both sides of the suction valve 1 are provided with a circular mouth 11 symmetrical to each other. The rear end of the suction valve 1 is provided with a first connecting tube 12. At a front end opposing to the position of the first connecting tube 12, a second connecting tube 13 is provided. The diameter of the mouth of the second connecting tube 13 is larger than that of the body of the suction valve 1. The bottom of the inner edge of the second connecting tube 13 is provided with an annular chamfer 131 (FIG. 2) at the position connecting to the body of the suction valve 1. A recessed region 132 extends outwardly from the inner edge of the second connecting tube 13 and is provided at a middle position thereof. The second connecting tube 13 is used to connect a circular rotating tube 2. The outer periphery of the rotating tube 2 is provided with a protruding ring 21. The protruding ring 21 is so resilient that the periphery of the mouth of the bottom of the rotating tube 2 exactly abuts against the chamfer 131 within the second connecting tube 13 when the rotating tube 2 is received in the second connecting tube 13. Meanwhile, the outer periphery of the protruding ring 21 of the rotating tube 2 is exactly received in the recessed region 132 of the second connecting tube 13, as shown in FIG. 2. As a result, a slight gap is formed between the outer surface of the rotating tube 2 and the inner edge of the second connecting tube 13. Finally, the second connecting tube 13 is connected with a hollow sleeve 3 in a manner that the sleeve 3 is disposed onto the outer circumferential surface of the rotating tube 2. The circumferential wall of the sleeve 3 is received in the gap formed between the recessed region 132 and the rotating tube 2, thereby to fix the position of the rotating tube 2 without sliding outwardly. FIG. 3 shows the state in which the present invention is completely assembled. Further, the tube body of the second connecting tube 13 is provided with an injection hole 14 thereon whereby an operator can inject medicines therein easily.

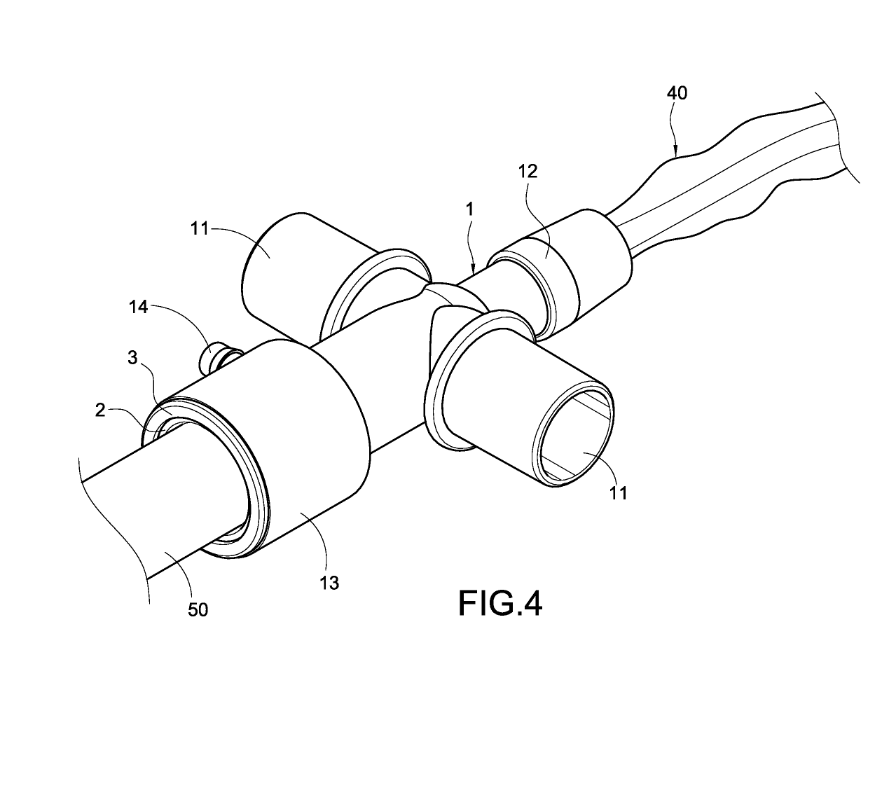

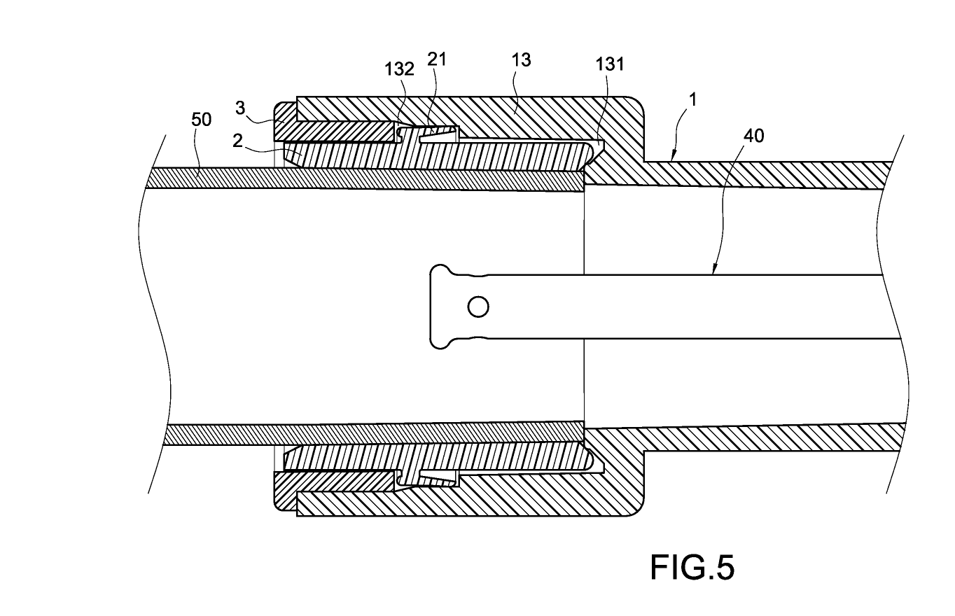

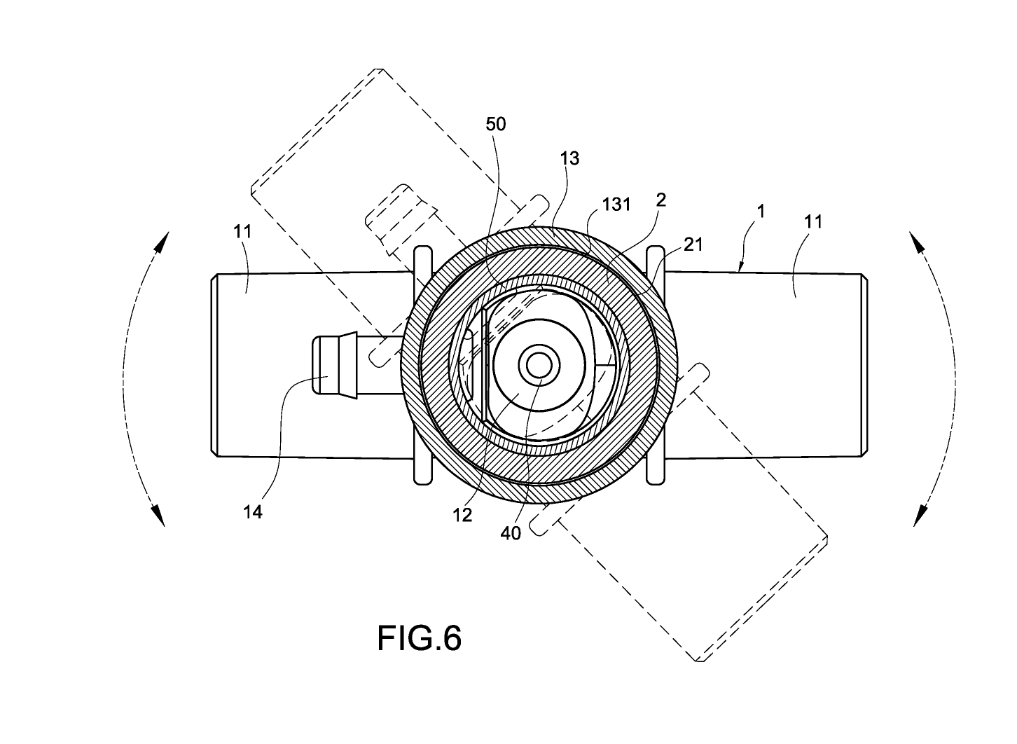

FIG. 4 is an assembled perspective view of the present invention. FIG. 5 is an assembled cross-sectional view of the present invention. FIG. 6 is a schematic view showing the operation of the present invention. As shown in FIG. 4, in the suction valve 1 of the present invention, the first connecting tube 12 is connected with a conduit 40, and the second connecting tube 13 is connected with a connecting tube 50 for a patient (not shown). The connecting tube 50 is disposed in the rotating tube 2 within the second connecting tube 13, as shown in the cross-sectional view of FIG. 5. Since the rotating tube 2 is provided within the second connecting tube 13 and the sleeve 3 is used to fix the position of the rotating tube 2 without sliding outwardly, the protruding ring 21 provided on the outer periphery of the rotating tube 2 abuts within the recessed region 132, thereby to form a substantial gap between the rotating tube 2 and the inner edge of the second connecting tube 13. The suction tube 1 and the rotating tube 2 can be displaced with respect to each other. Therefore, after the rotating tube 2 is connected to the connecting tube 50, if the user intends to adjust the position of the connecting tube of the suction tube, he/she may directly rotate the suction valve 1 to produce a mutual displacement between the suction valve 1 and the connecting tube 50 and thus effect a rotation, as shown in arrows of FIG. 6. In this way, the problem existed in prior art that the connecting tube 50 should be removed first to allow the adjustment of the position of the suction valve is avoided. Further, a leftward or rightward rotation is possible according to the demands for different adjustments, thereby to increase the convenience in using the suction valve.

Although the present invention has been described with reference to the foregoing preferred embodiment, it will be understood that the invention is not limited to the details thereof. Various equivalent variations and modifications can still be occurred to those skilled in this art in view of the teachings of the present invention. Thus, all such variations and equivalent modifications are also embraced within the scope of the invention as defined in the appended claims.

What is claimed is:

1. A rotating means for a suction tube, comprising:
a suction valve, the interior thereof communicating with each other, an end of the suction valve having a first connecting tube, a second connecting tube provided at the other end of the suction valve opposing to the first connecting tube, an annular chamfer provided at the connecting portion of the second connecting tube and the suction valve, an annular recessed region extending outwardly from a middle position of the second connecting tube;
a rotating tube disposed within the second connecting tube, the bottom end of the rotating tube exactly abutting against the chamfer, the outer circumferential surface of the rotating tube provided with a protruding ring, the protruding ring being resilient so as to abut against the interior of the second connecting tube; and
a sleeve connected with the second connecting tube and disposed on the outer circumferential surface of the rotating tube, thereby to fix the position of the rotating tube within the second connecting tube,
wherein the protruding ring provided on the rotating tube abuts against the interior of the second connecting tube, a gap is formed between the outer circumferential surface of the rotating tube and the interior of the second connecting tube, thereby to produce a mutual displacement between the rotating tube and the interior of the second connecting tube.

2. The rotating means for a suction tube according to claim 1, wherein the protruding ring abuts within the recessed region.

3. The rotating means for a suction tube according to claim 1, wherein both sides of the first connecting tube and the second connecting tube of the suction valve are provided with corresponding mouths.

4. The rotating means for a suction tube according to claim 1, wherein the profile of the suction valve is formed into a cross.

5. The rotating means for a suction tube according to claim 1, wherein the body of the second connecting tube is provided with an injection hole.
